SEQUENCE LISTING (4 sequences)

SEQ ID NO: 1 - DNA, 576 bp, Homo sapiens
atgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtga

SEQ ID NO: 2 - DNA, 4071 bp, Homo Sapiens
atggagagcaaggtgctgctggccgtcgccctgtggctctgcgtggagacccgggccgcctctgtgggtttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccccagattggtgagaaatctctaatctctcctgtggattcctaccagtacggcaccactcaaacgctgacatgtacggtctatgccattcctcccccgcatcacatccactggtattggcagttggaggaagagtgcgccaacgagcccagccaagctgtctcagtgacaaacccatacccttgtgaagaatggagaagtgtggaggacttccagggaggaaataaaattgaagttaataaaaatcaatttgctctaattgaaggaaaaaacaaaactgtaagtacccttgttatccaagcggcaaatgtgtcagctttgtacaaatgtgaagcggtcaacaaagtcgggagaggagagagggtgatctccttccacgtgaccaggggtcctgaaattactttgcaacctgacatgcagcccactgagcaggagagcgtgtctttgtggtgcactgcagacagatctacgtttgagaacctcacatggtacaagcttggcccacagcctctgccaatccatgtgggagagttgcccacacctgtttgcaagaacttggatactctttggaaattgaatgccaccatgttctctaatagcacaaatgacattttgatcatggagcttaagaatgcatccttgcaggaccaaggagactatgtctgccttgctcaagacaggaagaccaagaaaagacattgcgtggtcaggcagctcacagtcctagagcgtgtggcacccacgatcacaggaaacctggagaatcagacgacaagtattggggaaagcatcgaagtctcatgcacggcatctgggaatccccctccacagatcatgtggtttaaagataatgagacccttgtagaagactcaggcattgtattgaaggatgggaaccggaacctcactatccgcagagtgaggaaggaggacgaaggcctctacacctgccaggcatgcagtgttcttggctgtgcaaaagtggaggcatttttcataatagaaggtgcccaggaaaagacgaacttggaaatcattattctagtaggcacggcggtgattgccatgttcttctggctacttcttgtcatcatcctacggaccgttaagcgggccaatggaggggaactgaagacaggctacttgtccatcgtcatggatccagatgaactcccattggatgaacattgtgaacgactgccttatgatgccagcaaatgggaattccccagagaccggctgaagctaggtaagcctcttggccgtggtgcctttggccaagtgattgaagcagatgcctttggaattgacaagacagcaacttgcaggacagtagcagtcaaaatgttgaaagaaggagcaacacacagtgagcatcgagctctcatgtctgaactcaagatcctcattcatattggtcaccatctcaatgtggtcaaccttctaggtgcctgtaccaagccaggagggccactcatggtgattgtggaattctgcaaatttggaaacctgtccacttacctgaggagcaagagaaatgaatttgtcccctacaagaccaaaggggcacgattccgtcaagggaaagactacgttggagcaatccctgtggatctgaaacggcgcttggacagcatcaccagtagccagagctcagccagctctggatttgtggaggagaagtccctcagtgatgtagaagaagaggaagctcctgaagatctgtataaggacttcctgaccttggagcatctcatctgttacagcttccaagtggctaagggcatggagttcttggcatcgcgaaagtgtatccacagggacctggcggcacgaaatatcctcttatcggagaagaacgtggttaaaatctgtgactttggcttggcccgggatatttataaagatccagattatgtcagaaaaggagatgctcgcctccctttgaaatggatggccccagaaacaatttttgacagagtgtacacaatccagagtgacgtctggtcttttggtgttttgctgtgggaaatattttccttaggtgcttctccatatcctggggtaaagattgatgaagaattttgtaggcgattgaaagaaggaactagaatgagggcccctgattatactacaccagaaatgtaccagaccatgctggactgctggcacggggagcccagtcagagacccacgttttcagagttggtggaacatttgggaaatctcttgcaagctaatgctcagcaggatggcaaagactacattgttcttccgatatcagagactttgagcatggaagaggattctggactctctctgcctacctcacctgtttcctgtatggaggaggaggaagtatgtgaccccaaattccattatgacaacacagcaggaatcagtcagtatctgcagaacagtaagcgaaagagccggcctgtgagtgtaaaaacatttgaagatatcccgttagaagaaccagaagtaaaagtaatcccagatgacaaccagacggacagtggtatggttcttgcctcagaagagctgaaaactttggaagacagaaccaaattatctccatcttttggtggaatggtgcccagcaaaagcagggagtctgtggcatctgaaggctcaaaccagacaagcggctaccagtccggatatcactccgatgacacagacaccaccgtgtactccagtgaggaagcagaacttttaaagctgatagagattggagtgcaaaccggtagcacagcccagattctccagcctgactcggggaccacactgagctctcctcctgtttaa

SEQ ID NO: 3 - RNA, 19 nt, Homo Sapiens
  Sense strand of Cand5 siRNA
accucaccaaggccagcac

SEQ ID NO: 4 - RNA, 19 nt, Homo Sapiens
  Antisense strand for Cand5 siRNA
gugcuggccuuggugaggu

(19) United States
(12) Patent Application Publication
(10) Pub. No.: US 2009/0226531 A1
(43) Pub. Date: Sep. 10, 2009

(54) METHODS AND COMPOSITION FOR INTRAOCULAR DELIVERY OF THERAPEUTIC SIRNA

(75) Inventors: Robert T. LYONS, Laguna Hills, CA (US); Hongwen Ma, San Diego, CA (US)

Correspondence Address: ALLERGAN, INC., 2525 DUPONT DRIVE, T2-7H, IRVINE, CA, 92612-1599, US

(73) Assignee: ALLERGAN, INC., Irvine, CA (US)

(21) Appl. No.: 12/044,889

(22) Filed: Mar. 7, 2008

(51) Int. Cl. A61K 31/7105; A61K 9/14; A61P 27/02

(52) U.S. Cl. 424/501; 977/773; 514/44 A

(57) ABSTRACT

Biocompatible intraocular drug delivery systems include nanoparticles that encapsulate siRNA molecules. The drug delivery systems may be placed in an eye to treat or reduce the occurrence of one or more ocular conditions, such as retinal damage, including glaucoma and proliferative vitreoretinopathy among others.

### BACKGROUND

The present invention generally relates to compositions, drug delivery systems and methods to treat an eye of a patient, and more specifically to drug delivery systems comprising short interfering ribonucleic acid (siRNA) molecules encapsulated in nanoparticles, and to methods of making and using such systems, for example, to treat or reduce one or more symptoms of an ocular condition to improve or maintain vision of a patient.

RNA has been used for several years to reduce or interfere with expression of targeted genes in a variety of systems. Although originally thought to require use of long double-stranded RNA (dsRNA) molecules, the active mediators of RNAi are now known to be short dsRNAs. Short single-stranded antisense RNA molecules were demonstrated to be effective inhibitors of gene expression more than a decade ago, but are susceptible to degradation by a variety of nucleases and are therefore of limited utility without chemical modification. Double-stranded RNAs are surprisingly stable and, unlike single-stranded DNA or antisense RNA oligonucleotides, do not need extensive modification to survive in tissue culture media or living cells.

Short interfering RNAs are naturally produced by degradation of long dsRNAs by Dicer, an RNase III class enzyme. While these fragments are usually about 21 bases long, synthetic dsRNAs of a variety of lengths, ranging from 18 bases to 30 bases (D. -H. Kim et al., Synthetic dsRNA dicer-substrates enhance RNAI potency and efficacy, 23 Nature Biotechnology 222-226 (2005)), can be used to suppress gene expression. These short dsRNAs are bound by the RNA Induced Silencing Complex (RISC), which contains several protein components including a ribonuclease that degrades the targeted mRNA. The antisense strand of the dsRNA directs target specificity of the RISC RNase activity, while the sense strand of an RNAi duplex appears to function mainly to stabilize the RNA prior to entry into RISC and is degraded or discarded after entering RISC.

Chemically synthesized RNAi duplexes have historically been made as two 21-mer oligonucleotides that form a 19-base RNA duplex with two deoxythymidine bases added as 3′overhangs. (S. M. Elbashir et al., Functional anatomy of siRNAs for mediating efficient RNAI in Drosophila melanogaster embryo lysate, 20 EMBO J. 6877-6888 (2001)). Blunt 19-mer duplexes can also be used to trigger RNAi in mammalian systems. (F. Czaudema, Structural variations and stabilizing modifications of synthetic siRNAs in mammalian cells, 31 Nucleic Acids Res. 2705-2716 (2003)). These blunt duplexes, however, are generally less potent. Blunt duplexes can be effectively used for longer RNAs that are Dicer substrates. D. -H. Kim et al., supra. In this case, the duplex is processed by Dicer to 21-mer length with 2-base 3′-overhangs before entry into RISC.

Relatively recently, researchers observed that double stranded RNA (“dsRNA”) could be used to inhibit protein expression. This ability to silence a gene has broad potential for treating human diseases, and many researchers and commercial entities are currently investing considerable resources in developing therapies based on this technology.

It is generally considered that the major mechanism of RNA induced silencing (RNA interference, or RNAi) in mammalian cells is mRNA degradation. Initial attempts to use RNAi in mammalian cells focused on the use of long strands of dsRNA. However, these attempts to induce RNAi met with limited success, due in part to the induction of the interferon response, which results in a general, as opposed to a target-specific, inhibition of protein synthesis. Thus, long dsRNA is not a viable option for RNAi in mammaliansystems.

More recently it has been shown that when short (18-30 bp) RNA duplexes are introduced into mammalian cells in culture, sequence-specific inhibition of target mRNA can be realized without inducing an interferon response. Certain of these short dsRNAs, referred to as small inhibitory RNAs (“siRNAs”), can act catalytically at sub-molar concentrations to cleave greater than 95% of the target mRNA in the cell. A description of the mechanisms for siRNA activity, as well as some of its applications are described in Provost et al. (2002) Ribonuclease Activity and RNA Binding of Recombinant Human Dicer, EMBO J. 21(21): 5864-5874; Tabara et al. (2002).

From a mechanistic perspective, introduction of long double stranded RNA into plants and invertebrate cells is broken down into siRNA by a Type III endonuclease known as Dicer. Sharp, RNA interference—2001, Genes Dev. 2001, 15:485. Dicer, a ribonuclease-III-like enzyme, processes the dsRNA into 19-23 base pair short interfering RNAs with characteristic two base 3′ overhangs. Bernstein, Caudy, Hammond, & Hannon (2001) Role for a bidentate ribonuclease in the initiation step of RNA interference, Nature 409:363. The siRNAs are then incorporated into an RNA-induced silencing complex (RISC) where one or more helicases unwind the siRNA duplex, enabling the complementary antisense strand to guide target recognition. Nykanen, Haley, & Zamore (2001) ATP requirements and small interfering RNA structure in the RNA interference pathway, Cell 107:309. Upon binding to the appropriate target mRNA, one or more endonucleases within the RISC cleaves the target to induce silencing. (Elbashir, Lendeckel, & Tuschl (2001) RNA interference is mediated by 21- and 22-nucleotide RNAs, Genes Dev. 15:188, FIG. 1).

The interference effect can be long lasting and may be detectable after many cell divisions. Moreover, RNAi exhibits sequence specificity. Kisielow, M. et al. (2002) Isoform-specific knockdown and expression of adaptor protein ShcA using small interfering RNA, J. Biochem. 363:1-5. Thus, the RNAi machinery can specifically knock down one type of transcript, while not affecting closely related mRNA. These properties make siRNA a potentially valuable tool for inhibiting gene expression and studying gene function and drug target validation. Moreover, siRNAs are potentially useful as therapeutic agents against: (1) diseases that are caused by over-expression or misexpression of genes; and (2) diseases brought about by expression of genes that contain mutations.

### SUMMARY

The present invention provides new drug delivery systems, and methods of making and using such systems, for administering siRNA molecules to an eye, for example, to achieve one or more desired therapeutic effects. The drug delivery systems are in the form of nanoparticles encapsulating the siRNA molecules, wherein the nanoparticles are comprised of biodegradable polymers, biodegradable co-polymers, or combinations thereof, and wherein the nanoparticles may be administered in an aqueous suspension or in a viscoelastic hydrogel.

Intraocular drug delivery systems in accordance with the disclosure herein comprise a therapeutic component and a drug release sustaining component associated with the therapeutic component. The therapeutic component comprises at least one siRNA molecule, and the drug release sustaining component comprises a biodegradable polymer, a biodegradable co-polymer, or combinations thereof.

The polymeric component of the present systems may comprise a polymer and/or a copolymer selected from the group consisting of poly-lactic acid (PLA), poly-glycolic acid (PGA), poly-lactide-co-glycolide (PLGA) (e.g. R203H), polyesters, poly (ortho ester), poly(phosphazine), poly (phosphate ester), polyethylene glycol (PEG), triblock copolymers polycaprolactones, gelatin, collagen, poly(D,L-lysine), derivatives thereof, and combinations thereof.

A method of making the present systems involves encapsulating, combining or mixing the therapeutic component with the polymeric component to form a mixture. The mixture may then be extruded or compressed to form a single composition. The single composition may then be processed to form individual nanoparticles suitable for placement in an eye of a patient. The nanoparticles may be formulated as an aqueous suspension comprising particles and the nanoparticles may further be contained in and administered in a viscoelastic gel. The particles of the aqueous suspension may have diameters of 10 nm-2000 nm, 50 nm-1000 nm, 100 nm-200 nm or a combination thereof. The viscoelastic gel of the aqueous suspension may be comprised of a polysaccharide, such as hyaluronic acid, or combinations thereof. The viscoelastic hydrogels of the invention may comprise 5% -30% w:w nanoparticles and may comprise 1%-5% hyaluronic acid. The viscoelastic hydrogel may additionally comprise a buffer and may be isotonic.

The nanoparticles, aqueous suspension, viscoelastic hydrogels and combinations thereof of the drug delivery systems of the invention may be placed in an ocular region to treat a variety of ocular conditions, such as treating, preventing, or reducing at least one symptom associated with glaucoma, or ocular conditions related to excessive excitatory activity or glutamate receptor activation. Placement of the drug delivery systems of the present invention may be through injection via a needle.

Kits in accordance with the present invention may comprise one or more of the present systems, and instructions for using the systems. For example, the instructions may explain how to administer the present drug delivery systems to a patient, and types of conditions that may be treated with the systems.

Each and every feature described herein, and each and every combination of two or more of such features, is included within the scope of the present invention provided that the features included in such a combination are not mutually inconsistent. In addition, any feature or combination of features may be specifically excluded from any embodiment of the present invention.

Additional aspects and advantages of the present invention are set forth in the following description, examples, and claims, particularly when considered in conjunction with the accompanying drawings.

### DESCRIPTION

As described herein, the use of one or more intraocular drug delivery systems, such as the nanoparticles (NPs) containing siRNA molecules encapsulated within the NPs, may effectively treat one or more undesirable ocular conditions. The present drug delivery system comprises NPs comprised of one or more biodegradable polymers and/or co-polymers, which may be administered in the form of an aqueous suspension, or in a viscoelastic gel.

### 1. DEFINITIONS

For the purposes of this description, we use the following terms as defined in this section, unless the context of the word indicates a different meaning.

As used herein, an “intraocular drug delivery system” refers to a device or element that is structured, sized, or otherwise configured to be placed in an eye. The present drug delivery systems are generally biocompatible with physiological conditions of an eye and do not cause unacceptable or undesirable adverse side effects. The present drug delivery systems may be placed in an eye without disrupting vision of the eye. The present drug delivery system comprises a plurality of nanoparticles.

As used herein, a “therapeutic component” refers to a portion of a drug delivery system comprising one or more therapeutic agents, active ingredients, or substances used to treat a medical condition of the eye. The therapeutic component is typically homogenously distributed throughout the nanoparticles. The therapeutic agents of the therapeutic component are typically ophthalmic ally acceptable, and are provided in a form that does not cause adverse reactions when the implant is placed in an eye. As discussed herein, the therapeutic agents can be released from the drug delivery systems in a biologically active form. For example, the therapeutic agents may retain their three dimensional structure when released from the system into an eye.

As used herein, a “drug release sustaining component” refers to a portion of the drug delivery system that is effective in providing a sustained release of the therapeutic agents of the systems. A drug release sustaining component may be a biodegradable polymer matrix, or it may be a coating covering a core region of a nanoparticle that comprises a therapeutic component.

As used herein, “associated with” means mixed with, dispersed within, coupled to, covering, or surrounding.

As used herein, an “ocular region” or “ocular site” refers generally to any area of the eyeball, including the anterior and posterior segment of the eye, and which generally includes, but is hot limited to, any functional (e.g., for vision) or structural tissues found in the eyeball, or tissues or cellular layers that partly or completely line the interior or exterior of the eyeball. Specific examples of areas of the eyeball in an ocular region include the anterior chamber, the posterior chamber, the vitreous cavity, the choroid, the suprachoroidal space, the subretinal space, the conjunctiva, the subconjunctival space, the episcleral space, the intracorneal space, the epicorneal space, the sclera, the pars plana, surgically-induced avascular regions, the macula, and the retina.

As used herein, an “ocular condition” is a disease, ailment or condition which affects or involves the eye or one of the parts or regions of the eye. Broadly speaking the eye includes the eyeball and the tissues and fluids which constitute the eyeball, the periocular muscles (such as the oblique and rectus muscles) and the portion of the optic nerve which is within or adjacent to the eyeball.

An anterior ocular condition is a disease, ailment or condition which affects or which involves an anterior (i.e. front of the eye) ocular region or site, such as a periocular muscle, an eye lid or an eye ball tissue or fluid which is located anterior to the posterior wall of the lens capsule or ciliary muscles. Thus, an anterior ocular condition primarily affects or involves the conjunctiva, the cornea, the anterior chamber, the iris; the posterior chamber (behind the iris, but in front of the posterior wall of the lens capsule), the lens or the lens capsule and blood vessels and nerve which vascularize or innervate an anterior ocular region or site.

Thus, an anterior ocular condition can include a disease, ailment or condition, such as for example, aphakia; pseudophakia; astigmatism; blepharospasm; cataract; conjunctival diseases; conjunctivitis; corneal diseases;, corneal ulcer; dry eye syndromes; eyelid diseases; lacrimal apparatus diseases; lacrimal duct obstruction; myopia; presbyopia; pupil disorders; refractive disorders and strabismus. Glaucoma can also be considered to be an anterior ocular condition because a clinical goal of glaucoma treatment can be to reduce a hypertension of aqueous fluid in the anterior chamber of the eye (i.e. reduce intraocular pressure).

A posterior ocular condition is a disease, ailment or condition which primarily affects or involves a posterior ocular region or site such as choroid or sclera (in a position posterior to a plane through the posterior wall of the lens capsule), vitreous, vitreous chamber, retina, retinal pigmented epithelium, Bruch's membrane, optic nerve (i.e. the optic disc), and blood vessels and nerves which vascularize or innervate a posterior ocular region or site.

Thus, a posterior ocular condition can include a disease, ailment or condition, such as for example, acute macular neuroretinopathy; Behcet's disease; choroidal neovascularization; diabetic uveitis; histoplasmosis; infections, such as fungal or viral-caused infections; macular degeneration, such as acute macular degeneration, non-exudative age related macular degeneration and exudative age related macular degeneration; edema, such as macular edema, cystoid macular edema and diabetic macular edema; multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial occlusive disease, retinal detachment, uveitic retinal disease; sympathetic opthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, non-retinopathy diabetic retinal dysfunction, retinitis pigmentosa, and glaucoma. Glaucoma can be considered a posterior ocular condition because the therapeutic goal is to prevent the loss of or reduce the occurrence of loss of vision due to damage to or loss of retinal cells or optic nerve cells (i.e. neuroprotection).

The term “biodegradable polymer” refers to a polymer or polymers which degrade in vivo, and wherein erosion of the polymer or polymers over time occurs concurrent with or subsequent to release of the therapeutic agent. Specifically, hydrogels such as methylcellulose which act to release drug through polymer swelling are specifically excluded from the term “biodegradable polymer”. The terms “biodegradable” and “bioerodible” are equivalent and are used interchangeably herein. A biodegradable polymer may be a homopolymer, a copolymer, or a polymer comprising more than two different polymeric units.

The term “treat”, “treating”, or “treatment” as used herein, refers to reduction or resolution or prevention of an ocular condition, ocular injury or damage, or to promote healing of injured or damaged ocular tissue. The term “therapeutically effective amount” as used herein, refers to the level or amount of agent needed to treat an ocular condition, or reduce or prevent ocular injury or damage without causing significant negative or adverse side effects to the eye or a region of the eye. Intraocular drug delivery systems have been developed which can release drug loads over various' time periods. These systems, which when placed into an eye of an individual, such as the vitreous of an eye, provide therapeutic levels of a macromolecule therapeutic agent for extended periods of time (e.g., for about one week or more). In certain embodiments, the macromolecule therapeutic agent is an siRNA having at least one property selected from the group consisting of anti-angiogenesis, ocular hemorrhage treatment, non-steroidal anti-inflammatory, growth factor (e.g. VEGF) inhibitor, growth factor, cytokines and antibiotics. The disclosed systems are effective in treating ocular conditions, such as posterior ocular conditions, such as glaucoma and neovascularization, and generally improving or maintaining vision in an eye.

The phrase “gene silencing” refers to a process by which the expression of a specific gene product is lessened or attenuated. Gene silencing can take place by a variety of pathways. Unless specified otherwise, as used herein, gene silencing refers to decreases in gene product expression that results from RNA interference (RNAi), a defined, though partially characterized pathway whereby small inhibitory RNA (siRNA) act in concert with host proteins (e.g., the RNA induced silencing complex, RISC) to degrade messenger RNA (mRNA) in a sequence-dependent fashion. The level of gene silencing can be measured by a variety of means, including, but not limited to, measurement of transcript levels by Northern Blot Analysis, B-DNA techniques, transcription-sensitive reporter constructs, expression profiling (e.g., DNA chips), and related technologies. Alternatively, the level of silencing can be measured by assessing the level of the protein encoded by a specific gene. This can be accomplished by performing a number of studies including Western Analysis, measuring the levels of expression of a reporter protein that has e.g., fluorescent properties (e.g., GFP) or enzymatic activity (e.g., alkaline phosphatases), or several other procedures.

The term “siRNA” refers to small inhibitory RNA duplexes that induce the RNA interference (RNAi) pathway. These molecules can vary in length (generally 18-30 base pairs) and contain varying degrees of complementarity to their target mRNA in the antisense strand. Some, but not all, siRNA have unpaired overhanging bases on the 5′ or 3′ end of the sense strand and/or the antisense strand. The term “siRNA” includes duplexes of two separate strands, as well as single strands that can form hairpin structures comprising a duplex region.

### 2. COMPONENTS OF THE DRUG DELIVERY SYSTEM

2.1 The Therapeutic Component

As noted above, the therapeutic component of the drug delivery system comprises at least one siRNA molecule. Various types and kinds of siRNA molecules are per se known to those skilled in the art, and known for treatment of various biologincal and pharmacological conditions. siRNA molecules may be divided into five (5) groups (non-functional, semi-functional, functional, highly functional, and hyper-functional) based on the level or degree of silencing that they induce in cultured cell lines. As used herein, these definitions are based on a set of conditions where the siRNA is transfected into said cell line at a concentration of 100 nM and the level of silencing is tested at a time of roughly 24 hours after transfection, and not exceeding 72 hours after transfection. In this context, “non-functional siRNA” are defined as those siRNA that induce less than 50% (<50%) target silencing. “Semi-functional siRNA” induce 50-79% target silencing. “Functional siRNA” are molecules that induce 80-95% gene silencing. “Highly-functional siRNA” are molecules that induce greater than 95% gene silencing. “Hyperfunctional siRNA” are a special class of molecules. For purposes of this document, hyperfunctional siRNA are defined as those molecules that: (1) induce greater than 95% silencing of a specific target when they are transfected at subnanomolar concentrations (i.e., less than one nanomolar); and/or (2) induce functional (or better) levels of silencing for greater than 96 hours. These relative functionalities (though not intended to be absolutes) may be used to compare siRNAs to a particular target for applications such as functional genomics, target identification and therapeutics.

In some preferred embodiments of the present drug delivery systems, the siRNA has a nucleotide sequence that is effective in inhibiting cellular production of vascular endothelial growth factor (VEGF) or VEGF receptors. VEGF is a endothelial cell mitogen (Connolly D. T. , et al., Tumor vascular permeability factor stimulates endothelial cell growth and angiogenesis. J. Clin. Invest. 84: 1470- 1478 (1989)), that through binding with its receptor, VEGFR, plays an important role in the growth and maintenance of vascular endothelial cells and in the development of new blood- and lymphatic-vessels (Aiello L. P. , et al., Vascular endothelial growth factor in ocular fluid of patients with diabetic retinopathy and other retinal disorders, New Engl. J. Med. 331: 1480- 1487 (1994)).

Currently, the VEGF receptor family is believed to consist of three types of receptors, VEGFR-1 (Fit-1), VEGFR-2 (KDR/Flk-1) and VEGFR-3 (Flt-4), all of which belong to the receptor type tyrosine kinase superfamily (Mustonen T. et al., Endothelial receptor tyrosine kinases involved in angiogenesis, J. Cell Biol. 129: 895-898 (1995)). Among these receptors, VEGFR-1 appears to bind the strongest to VEGF, VEGFR-2 appears to bind more weakly than VEGFR-1, and VEGFR-3 shows essentially no binding, although it does bind to other members of the VEGF family. The tyrosine kinase domain of VEGFR-1, although much weaker than that of VEGFR-2, tranduces signals for endothelial cells. Thus, VEGF is a substance that stimulates the growth of new blood vessels. The development of new blood vessels, neovascularization or angiogenesis, in the eye is believed to cause loss of vision in wet macular degeneration and other ocular conditions, including edema.

Sustained release drug delivery systems which include active siRNA molecules can release effective amounts of active siRNA molecules that associate with a ribonuclease complex (RISC) in target cells to inhibit the production of a target protein, such as VEGF or VEGF receptors. The siRNA of the present systems can be double-stranded or single stranded RNA molecules and may have a length less than about 50 nucleotides, less than about 40 nucleotides, less than about 30 nucleotides, less than about 20 nucleotides or less than 10 nucleotides. In certain embodiments, the systems may comprise a siRNA having a hairpin structure, and thus may be understood to be a short hairpin RNA (shRNA), as available from Invitrogen (San Diego, Calif.).

Some siRNAs that are used in the present systems preferably inhibit production of VEGF or VEGF receptors compared to other cellular proteins. In certain embodiments, the siRNAs can inhibit production of VEGF or VEGFR by at least 50%, preferably by at least 60%, and more preferably by about 70% or more. Thus, these siRNAs have nucleotide sequences that are effective in providing these desired ranges of inhibition.

The nucleotide sequence of the human VEGF isoform, VEGF 165 is identified as SEQ ID NO: 1, below. The nucleotide sequence has a GenBank Accession Number AB021221.

(SEQ ID NO:1)

atgaactttctgctgtcttgggtgcattggagccttgccttgctgctcta
cctccaccatgccaagtggtcccaggctgcacccatggcagaaggaggag
ggcagaatcatcacgaagtggtgaagttcatggatgtctatcagcgcagc
tactgccatccaatcgagaccctggtggacatcttccaggagtaccctga
tgagatcgagtacatcttcaagccatcctgtgtgcccctgatgcgatgcg
ggggctgctgcaatgacgagggcctggagtgtgtgcccactgaggagtcc
aacatcaccatgcagattatgcggatcaaacctcaccaaggccagcacat
aggagagatgagcttcctacagcacaacaaatgtgaatgcagaccaaaga
aagatagagcaagacaagaaaatccctgtgggccttgctcagagcggaga
aagcatttgtttgtacaagatccgcagacgtgtaaatgttcctgcaaaaa
cacagactcgcgttgcaaggcgaggcagcttgagttaaacgaacgtactt
gcagatgtgacaagccgaggcggtga

The nucleotide sequence of human VEGFR2 is identified as SEQ ID NO: 2, below. The nucleotide sequence has a GenBank Accession Number AF063658.

(SEQ ID NO:2)

atggagagcaaggtgctgctggccgtcgccctgtggctctgcgtggagac
ccgggccgcctctgtgggtttgcctagtgtttctcttgatctgcccaggc
tcagcatacaaaaagacatacttacaattaaggctaatacaactcttcaa
attacttgcaggggacagagggacttggactggctttggcccaataatca
gagtggcagtgagcaaagggtggaggtgactgagtgcagcgatggcctct
tctgtaagacactcacaattccaaaagtgatcggaaatgacactggagcc
tacaagtgcttctaccgggaaactgacttggcctcggtcatttatgtcta
tgttcaagattacagatctccatttattgcttctgttagtgaccaacatg
gagtcgtgtacattactgagaacaaaaacaaaactgtggtgattccatgt
ctcgggtccatttcaaatctcaacgtgtcactttgtgcaagatacccaga
aaagagatttgttcctgatggtaacagaatttcctgggacagcaagaagg
gctttactattcccagctacatgatcagctatgctggcatggtcttctgt
gaagcaaaaattaatgatgaaagttaccagtctattatgtacatagttgt
cgttgtagggtataggatttatgatgtggttctgagtccgtctcatggaa
ttgaactatctgttggagaaaagcttgtcttaaattgtacagcaagaact
gaactaaatgtggggattgacttcaactgggaatacccttcttcgaagca
tcagcataagaaacttgtaaaccgagacctaaaaacccagtctgggagtg
agatgaagaaatttttgagcaccttaactatagatggtgtaacccggagt
gaccaaggattgtacacctgtgcagcatccagtgggctgatgaccaagaa
gaacagcacatttgtcagggtccatgaaaaaccttttgttgcttttggaa
gtggcatggaatctctggtggaagccacggtgggggagcgtgtcagaatc
cctgcgaagtaccttggttacccacccccagaaataaaatggtataaaaa
tggaataccccttgagtccaatcacacaattaaagcggggcatgtactga
cgattatggaagtgagtgaaagagacacaggaaattacactgtcatcctt
accaatcccatttcaaaggagaagcagagccatgtggtctctctggttgt
gtatgtcccaccccagattggtgagaaatctctaatctctcctgtggatt
cctaccagtacggcaccactcaaacgctgacatgtacggtctatgccatt
cctcccccgcatcacatccactggtattggcagttggaggaagagtgcgc
caacgagcccagccaagctgtctcagtgacaaacccatacccttgtgaag
aatggagaagtgtggaggacttccagggaggaaataaaattgaagttaat
aaaaatcaatttgctctaattgaaggaaaaaacaaaactgtaagtaccct
tgttatccaagcggcaaatgtgtcagctttgtacaaatgtgaagcggtca
acaaagtcgggagaggagagagggtgatctccttccacgtgaccaggggt
cctgaaattactttgcaacctgacatgcagcccactgagcaggagagcgt
gtctttgtggtgcactgcagacagatctacgtttgagaacctcacatggt
acaagcttggcccacagcctctgccaatccatgtgggagagttgcccaca
cctgtttgcaagaacttggatactctttggaaattgaatgccaccatgtt
ctctaatagcacaaatgacattttgatcatggagcttaagaatgcatcct
tgcaggaccaaggagactatgtctgccttgctcaagacaggaagaccaag
aaaagacattgcgtggtcaggcagctcacagtcctagagcgtgtggcacc
cacgatcacaggaaacctggagaatcagacgacaagtattggggaaagca
tcgaagtctcatgcacggcatctgggaatccccctccacagatcatgtgg
tttaaagataatgagacccttgtagaagactcaggcattgtattgaagga
tgggaaccggaacctcactatccgcagagtgaggaaggaggacgaaggcc
tctacacctgccaggcatgcagtgttcttggctgtgcaaaagtggaggca
tttttcataatagaaggtgcccaggaaaagacgaacttggaaatcattat
tctagtaggcacggcggtgattgccatgttcttctggctacttcttgtca
tcatcctacggaccgttaagcgggccaatggaggggaactgaagacaggc
tacttgtccatcgtcatggatccagatgaactcccattggatgaacattg
tgaacgactgccttatgatgccagcaaatgggaattccccagagaccggc
tgaagctaggtaagcctcttggccgtggtgcctttggccaagtgattgaa
gcagatgcctttggaattgacaagacagcaacttgcaggacagtagcagt
caaaatgttgaaagaaggagcaacacacagtgagcatcgagctctcatgt
ctgaactcaagatcctcattcatattggtcaccatctcaatgtggtcaac
cttctaggtgcctgtaccaagccaggagggccactcatggtgattgtgga
attctgcaaatttggaaacctgtccacttacctgaggagcaagagaaatg
aatttgtcccctacaagaccaaaggggcacgattccgtcaagggaaagac
tacgttggagcaatccctgtggatctgaaacggcgcttggacagcatcac
cagtagccagagctcagccagctctggatttgtggaggagaagtccctca
gtgatgtagaagaagaggaagctcctgaagatctgtataaggacttcctg
accttggagcatctcatctgttacagcttccaagtggctaagggcatgga
gttcttggcatcgcgaaagtgtatccacagggacctggcggcacgaaata
tcctcttatcggagaagaacgtggttaaaatctgtgactttggcttggcc
cgggatatttataaagatccagattatgtcagaaaaggagatgctcgcct
ccctttgaaatggatggccccagaaacaatttttgacagagtgtacacaa
tccagagtgacgtctggtcttttggtgttttgctgtgggaaatattttcc
ttaggtgcttctccatatcctggggtaaagattgatgaagaattttgtag
gcgattgaaagaaggaactagaatgagggcccctgattatactacaccag
aaatgtaccagaccatgctggactgctggcacggggagcccagtcagaga
cccacgttttcagagttggtggaacatttgggaaatctcttgcaagctaa
tgctcagcaggatggcaaagactacattgttcttccgatatcagagactt
tgagcatggaagaggattctggactctctctgcctacctcacctgtttcc
tgtatggaggaggaggaagtatgtgaccccaaattccattatgacaacac
agcaggaatcagtcagtatctgcagaacagtaagcgaaagagccggcctg
tgagtgtaaaaacatttgaagatatcccgttagaagaaccagaagtaaaa
gtaatcccagatgacaaccagacggacagtggtatggttcttgcctcaga
agagctgaaaactttggaagacagaaccaaattatctccatcttttggtg
gaatggtgcccagcaaaagcagggagtctgtggcatctgaaggctcaaac
cagacaagcggctaccagtccggatatcactccgatgacacagacaccac
cgtgtactccagtgaggaagcagaacttttaaagctgatagagattggag
tgcaaaccggtagcacagcccagattctccagcctgactcggggaccaca
ctgagctctcctcctgtttaa

One specific example of a useful siRNA available from Acuity Pharmaceuticals (Pennsylvania) or Avecia Biotechnology under the name Cand5. Cand5 is a therapeutic agent that essentially silences the genes that produce VEGF. Thus, drug delivery systems including an siRNA selective for VEGF can prevent or reduce VEGF production in a patient in need thereof. The 5′ to 3′ nucleotide sequence of the sense strand of Cand5 is identified in SEQ ID NO: 3 below; and the 5′ to 3′ nucleotide sequence of the anti-sense strand of Cand5 is identified in SEQ ID NO: 4 below.

| | |
|---|---|
| ACCUCACCAAGGCCAGCACdTdT | (SEQ ID NO:3) |
| GUGCUGGCCUUGGUGAGGUdTdT | (SEQ ID NO:4) |

Another example of a useful siRNA available from Sirna Therapeutics (Colorado) under the name Sirna-027. Sirna-027 is a chemically modified short interfering RNA (siRNA) that targets vascular endothelial growth factor receptor-1 (VEGFR-1). Some additional examples of nucleic acid molecules that modulate the synthesis, expression and/or stability of an mRNA encoding one or more receptors of vascular endothelial growth factor are disclosed in U.S. Pat. No. 6,818,447 (Pavco). Sirna-027 has a sense strand with the sequence CUGAGUUUAAAAGGCACCCdTdT, and an antisense strand having the sequence GGGUGCCUUUUAAACUCAGdTdT)

Thus, the present drug delivery systems may comprise a VEGF or VEGFR inhibitor that includes an siRNA having a nucleotide sequence that is substantially identical to the nucleotide sequence of Cand5 or Sirna-027, identified above. For example, the nucleotide sequence of an siRNA may have at least about 80% sequence homology to the nucleotide sequence of Cand5 or Sirna-027 siRNAs. Preferably, a siRNA has a nucleotide sequence homology of at least about 90%, and more preferably at least about 95% of the Cand5 or Sirna-027 siRNAs. In other embodiments, the siRNA may have a homology to VEGF or VEGFR that results in the inhibition or reduction of VEGF or VEGFR synthesis.

The siRNA molecules can be contained in the drug delivery system (specifically encapsulated in the nanoparticles) either in free form or charge complexed with a cationic polymer. A cationic polymer is, in general, a polymer composed of positively charged macromolecule. Cationic polymers particularly cationic peptides have been shown to mediate transformation of RNA into cells. See, for example, WO/2006/046978. Suitable cationic polymers are, for example, a protoamine (a cationic peptide).

2.2 The Nanoparticles

As discussed herein, the polymeric component of the present systems comprises a biodegradable polymer, co-polymer, or combinations thereof, particularly as a plurality of biodegradable nanoparticles. Such particles may vary in shape. For example, certain embodiments of the present invention utilize substantially spherical particles. Other embodiments may utilize randomly configured particles, such as particles that have one or more flat or planar surfaces. The drug delivery system may comprise a population of such particles with a predetermined size distribution. For example, a major portion of the population may comprise particles having a desired diameter measurement.

The nanoparticles typically have an effective average particle size of less than about 400 nanometers, and in still further embodiments, a size less than about 200 nanometers, in a range of 100-200 nm, and with a low level of polydispersity.

The siRNA molecules are encapsulated in the nanoparticles either in free form or in complex with a cationic polymer, such as a protamine. Suitable cationic polymers include low molecular weight (about 50 to 150 kDa) or medium molecular weight (about 150 to 750 kDa) chitosan or chitosan derivatives; low molecular weight (about 50 to 150 kDa) or medium molecular weight (about 150 to 750 kDa) polypropylenimine dendrimers including generation 2; and low molecular weight (about 50 to 150 kDa) or medium molecular weight (about 150 to 750 kDa) block copolymers of poly(L-lysine) or polyethylenimine with polyethylene glycol (PEG). The molecular weight ranges of cationic polymers may be selected based on sufficient siRNA binding with low cytotoxicity. One suitable type of cationic polymer is a protamine, with are small, non-toxic cationic peptides, such as. siRNA molecules can be complexed with cationic polymers via procedures that are per se known.

Suitable polymeric materials or compositions for use in the nanoparticles include those materials which are compatible, that is biocompatible, with the eye so as to cause no substantial interference with the functioning or physiology of the eye. Such materials preferably include polymers that are at least partially and more preferably substantially completely biodegradable or bioerodible.

In addition to the foregoing, examples of useful polymeric materials include, without limitation, such materials derived from and/or including organic esters and organic ethers, which when degraded result in physiologically acceptable degradation products, including the monomers. Also, polymeric materials derived from and/or including, anhydrides, amides, orthoesters and the like, by themselves or in combination with other monomers, may also find use. The polymeric materials may be addition or condensation polymers, advantageously condensation polymers.

The polymeric materials may be cross-linked or non-cross-linked, for example not more than lightly cross-linked, such as less than about 5%, or less than about 1% of the polymeric material being cross-linked. For the most part, besides carbon and hydrogen, the polymers will include at least one of oxygen and nitrogen, advantageously oxygen. The oxygen may be present as oxy, e.g. hydroxy or ether, carbonyl, e.g. non-oxo-carbonyl, such as carboxylic acid ester, and the like. The nitrogen may be present as amide, cyano and amino. The polymers set forth in Heller, Biodegradable Polymers in Controlled Drug Delivery, In: CRC Critical Reviews in Therapeutic Drug Carrier Systems, Vol. 1, CRC Press, Boca Raton, Fla. 1987, pp 39-90, which describes encapsulation for controlled drug delivery, may find use in the present implants.

Of additional interest are polymers of hydroxyaliphatic carboxylic acids, either homopolymers or copolymers, and polysaccharides. Polyesters of interest include polymers of D-lactic acid, L-lactic acid, racemic lactic acid, glycolic acid, polycaprolactone, and combinations thereof. Generally, by employing the L-lactate or D-lactate, a slowly eroding polymer or polymeric material is achieved, while erosion is substantially enhanced with the lactate racemate.

Among the useful polysaccharides are, without limitation, calcium alginate, and functionalized celluloses, particularly carboxym ethyl cellulose esters characterized by being water insoluble, a molecular weight of about 5 kD to 500 kD, for example.

Other polymers of interest include, without limitation, polyesters, polyethers and combinations thereof which are biocompatible and may be biodegradable and/or bioerodible.

Some preferred characteristics of the polymers or polymeric materials for use in the present invention may include biocompatibility, compatibility with the therapeutic component, ease of use of the polymer in making the drug delivery systems of the present invention, a half-life in the physiological environment of at least about 6 hours, preferably greater than about one day, not significantly increasing the viscosity of the vitreous, and water insolubility.

The biodegradable polymeric materials which are included to form the matrix are desirably subject to enzymatic or hydrolytic instability. Water soluble polymers may be cross-linked with hydrolytic or biodegradable unstable cross-links to provide useful water insoluble polymers. The degree of stability can be varied widely, depending upon the choice of monomer, whether a homopolymer or copolymer is employed, employing mixtures of polymers, and whether the polymer includes terminal acid groups.

Also important to controlling the biodegradation of the polymer and hence the extended release profile of the drug delivery systems is the relative average molecular weight of the polymeric composition employed in the present systems. Different molecular weights of the same or different polymeric compositions may be included in the systems to modulate the release profile. In certain systems, the relative average molecular weight of the polymer will range from about 9 to about 64 kD, usually from about 10 to about 54 kD, and more usually from about 12 to about 45 kD.

In some drug delivery systems, copolymers of glycolic acid and lactic acid are used, where the rate of biodegradation is controlled by the ratio of glycolic acid to lactic acid. The most rapidly degraded copolymer has roughly equal amounts of glycolic acid and lactic acid. Homopolymers, or copolymers having ratios other than equal, are more resistant to degradation. The ratio of glycolic acid to lactic acid will also affect the brittleness of the system, where a more flexible system or implant is desirable for larger geometries. The % of polylactic acid in the polylactic acid polyglycolic acid (PLGA) copolymer can be 0-100%, preferably about 15-85%, more preferably about 35-65%. In some systems, a 50/50 PLGA copolymer is used.

The biodegradable polymer nanoparticles of the present systems may comprise a mixture of two or more biodegradable polymers. For example, the system may comprise a mixture of a first biodegradable polymer and a different second biodegradable polymer. One or more of the biodegradable polymers may have terminal acid groups.

Release of a drug from an erodible polymer is the consequence of several mechanisms or combinations of mechanisms. Some of these mechanisms include desorption from the implants surface, dissolution, diffusion through porous channels of the hydrated polymer and erosion. Erosion can be bulk or surface or a combination of both. It may be understood that the polymeric component of the present systems is associated with the therapeutic component so that the release of the therapeutic component into the eye is by one or more of diffusion, erosion, dissolution, and osmosis. As discussed herein, the matrix of an intraocular drug delivery system may release drug at a rate effective to sustain release of an amount of the therapeutic agent for more than one week after implantation into an eye. In certain systems, therapeutic amounts of the therapeutic agent are released for more than about one month, and even for about twelve months or more. For example, the therapeutic component can be released into the eye for a time period from about ninety days to about one year after the system is placed in the interior of an eye.

The release of the therapeutic agent from the intraocular systems comprising a biodegradable polymer matrix may include an initial burst of release followed by a gradual increase in the amount of the therapeutic agent released, or the release may include an initial delay in release of the therapeutic agent followed by an increase in release. When the system is substantially completely degraded, the percent of the therapeutic agent that has been released is about one hundred. Compared to existing implants, the systems disclosed herein do not completely release, or release about 100% of the therapeutic agent, until after about one week of being placed in an eye.

It may be desirable to provide a relatively constant rate of release of the therapeutic agent from the drug delivery system over the life of the system. For example, it may be desirable for the therapeutic agent to be released in amounts from about 0.01 pg to about 2 pg per day for the life of the system. However, the release rate may change to either increase or decrease depending on the formulation of the biodegradable polymer matrix. In addition, the release profile of the therapeutic agent may include one or more linear portions and/or one or more non-linear portions. Preferably, the release rate is greater than zero once the system has begun to degrade or erode. Thus, NPs can be made and/or combined using polymer blends to optimize release kinetics of the siRNA molecules.

As discussed in the examples herein, the present drug delivery systems comprise a therapeutic component and a polymeric component, as discussed above, which are associated to release an amount of the therapeutic siRNA agent that is effective in providing a concentration of the therapeutic agent in the vitreous of the eye for treating the desired condition, for example in a range from about 0.2 nM to about 5 pM. In addition or alternatively, the present systems can release a therapeutically effective amount of the siRNA molecule at a rate from about 0.003 pg/day to about 5000 pg/day. As understood by persons of ordinary skill in the art, the desired release rate and target drug concentration will vary depending on the particular therapeutic agent chosen for the drug delivery system, the ocular condition being treated, and the patient's health. Optimization of the desired target drug concentration and release rate can be determined using routine methods known to persons of ordinary skill in the art.

Drug delivery systems can be prepared where the center of the nanoparticles may be of one material and the surface may have one or more layers of the same or a different composition, where the layers may be cross-linked, or of a different molecular weight, different density or porosity, or the like. For example, where it is desirable to quickly release an initial bolus of drug, the center may be a polylactate coated with a polylactate-polyglycolate copolymer, so as to enhance the rate of initial degradation. Alternatively, the center may be polyvinyl alcohol coated with polylactate, so that upon degradation of the polylactate exterior the center would dissolve and be rapidly washed out of the eye.

The nanoparticles may be prepared and administered in the form of an aqueous suspension. The proportions of therapeutic agent, polymer, and any other modifiers may be empirically determined by formulating several implants, for example, with varying proportions of such ingredients. The therapeutic agent of the present systems is preferably from about 1% to 90% by weight of the drug delivery system. More preferably, the therapeutic agent is from about 20% to about 80% by weight of the system. In a preferred embodiment, the therapeutic agent comprises about 40% by weight of the system (e.g., 30%-50%). In another embodiment, the therapeutic agent comprises about 60% by weight of the system. A USP approved method for dissolution or release test can be used to measure the rate of release (USP 23; NF 18 (1995) pp. 17901798). For example, using the infinite sink method, a weighed sample of the implant is added to a measured volume of a solution containing 0.9% NaCl in water, where the solution volume will be such that the drug concentration is after release is less than 5% of saturation. The mixture is maintained at 37° C. and stirred slowly to maintain the particles in suspension. The appearance of the dissolved drug as a function of time may be followed by various methods known in the art, such as spectrophotometrically, HPLC, mass spectroscopy, etc. until the absorbance becomes constant or until greater than 90% of the drug has been released.

In addition to the therapeutic component, the intraocular drug delivery systems disclosed herein may include an excipient component, such as effective amounts of buffering agents, preservatives and the like. Suitable water soluble buffering agents include, without limitation, alkali and alkaline earth carbonates, phosphates, bicarbonates, citrates, borates, acetates, succinates and the like, such as sodium phosphate, citrate, borate, acetate, bicarbonate, carbonate and the like. These agents are advantageously present in amounts sufficient to maintain a pH of the system of between about 2 to about 9. and more preferably about 4 to about 8. As such the buffering agent may be as much as about 5% by weight of the total system. Suitable water soluble preservatives include sodium bisulfite, sodium bisulfate, sodium thiosulfate, ascorbate, benzalkonium chloride, chlorobutanol, thimerosal, phenylmercuric acetate, phenylmercuric borate, phenylmercuric nitrate, parabens, methylparaben, polyvinyl alcohol, benzyl alcohol, phenylethanol and the like and mixtures thereof. These agents may be present in amounts of from 0.001 to about 5% by weight and preferably 0.01 to about 2% by weight.

In some situations mixtures of drug delivery systems may be utilized employing the same or different pharmacological agents. In this way, a cocktail of release profiles, giving a biphasic or triphasic release with a single administration is achieved, where the pattern of release may be greatly varied.

In another embodiment, a delivery system comprises a biodegradable polymer, such as PLGA, and a VEGFNEGFR inhibitor. The system can be in the form of a population of biodegradable polymeric nanoparticles. The drug delivery system includes an amount of a VEGFNEGFR inhibitor that when released from the system, the inhibitor can provide a therapeutic effect. These drug delivery systems provide prolonged delivery of the VEGF inhibitor directly into the vitreous of an eye in need of treatment. Thus, these drug delivery systems can provide effective treatment of one or more ocular conditions, including without limitation, neovascularization, ocular tumors, and the like.

Embodiments of the present invention also relate to compositions comprising the present drug delivery systems. For example, and in one embodiment, a composition may comprise the present drug delivery system and an ophthalmically acceptable carrier component. Such a carrier component may be an aqueous composition, for example saline or a phosphate buffered liquid.

The present drug delivery systems are preferably administered to patients in a sterile form. For example, the present drug delivery systems, or compositions containing such systems, may be sterile when stored. Any routine suitable method of sterilization may be employed to sterilize the drug delivery-systems. For example, the present systems may be sterilized using radiation. Preferably, the sterilization method does not reduce the activity or biological or therapeutic activity of the therapeutic agents of the present systems, and lyophilization of the NPs of the invention may be employed to this end.

The drug delivery systems can be sterilized by gamma irradiation. As an example, the particles can be sterilized by 2.5 to 4.0 mrad of gamma irradiation. The particles can be terminally sterilized in their final primary packaging system including administration device e.g. syringe applicator. Alternatively, the particles can be sterilized alone and then aseptically packaged into an applicator system. In this case the applicator system can be sterilized by gamma irradiation, ethylene oxide (ETO), heat or other means. The drug delivery systems can be sterilized by gamma irradiation at low temperatures to improve stability or blanketed with argon, nitrogen or other means to remove oxygen. Beta irradiation or e-beam may also be used to sterilize the particles as well as UV irradiation. The dose of irradiation from any source can be lowered depending on the initial bioburden of the particles such that it may be much less than 2.5 to 4.0 mrad. The drug delivery systems may be manufactured under aseptic conditions from sterile starting components. The starting components may be sterilized by heat, irradiation (gamma, beta, UV), ETO or sterile filtration. Semi-solid polymers or solutions of polymers may be sterilized prior to drug delivery system fabrication and macromolecule incorporation by sterile filtration of heat. The sterilized polymers can then be used to aseptically produce sterile drug delivery systems.

2.3 The Hydrogels

The siRNA-containing nanoparticles may be prepared and administered as a long lasting suspension in a viscoelastic hydrogel. Hydrogels, especially injectable hydrogels, have been prepared from polysaccharides and their derivatives—particularly from hyaluronic acid, its salts and their mixtures—which have a zero, low or high degree of crosslinking. EP-A-0 161 887 thus describes the use of such injectable hydrogels for the treatment of arthritis. WO-A-96/33751 and WO-A-00/01428 describe injectable biphasic compositions whose continuous phase is based on such a hydrogel. Said continuous phase serves as an injection vehicle. See also WO/2000/016818 and WO/2005/112888.

In some modes of preparation, the hydrogel-forming composition comprises a macromer. Macromers include one or more “polymerizable group(s)” which generally refers to a chemical group that is polymerizable in the presence of free radicals. Polymerizable groups generally include a carbon-carbon double bond which can be an ethylenically unsaturated group or a vinyl group. Exemplary polymerizable groups include acrylate groups, methacrylate groups, ethacrylate groups, 2-phenyl acrylate groups, acrylamide groups, methacrylamide groups, itaconate groups, and styrene groups.

Polymers can be effectively derivatized in organic, polar, or anhydrous solvents, or solvent combinations to produce macromers. Generally, a solvent system is used that allows for polymer solubility and control over the derivatization with polymerizable groups. Polymerizable groups such as glycidyl acrylate can be added to polymers (including polysaccharides and polypeptides) in straightforward synthetic processes. In some aspects, the polymerizable group is present on the macromer at a molar ratio of 0.05 .mu.mol or greater of polymerizable group (such as an acrylate group) per 1 mg of macromer. In some aspects the macromer is derivatized with polymerizable groups in amount in the range from about 0.05 .mu.mol to about 2 .mu.mol of polymerizable group (such as an acrylate group) per 1 mg of macromer.

For example, a natural polymer such as hyaluronic acid can be reacted with a compound containing a polymerizable group, such as glycidyl acrylate, in the presence of formamide (and TEA, for pH control) to provide acrylate-derivatized hyaluronic acid molecules. The number and/or density of acrylate groups can be controlled using the present method, e.g., by controlling the relative concentration of reactive moiety to saccharide group content.

Crosslinker chemistry can also be used to add polymerizable groups to a polymer. For example, polymers can be derivatized with varying amounts of vinyl containing compounds such as vinylbenzoic acid. Polymer preparations can be mixed in the cold followed by the addition of a crosslinker such as 1-ethyl-3-(3-dimethylaminopropyl) carbodiimide.

In some aspects, the hydrogel-forming composition includes, as starting components, a higher molecular weight reactive polymer (macromers). Macromers generally include one or more reactive groups which allow them to become associated with each other following reaction of the reactive groups. In many cases macromers include polymerizable groups, such as ethylenically unsaturated groups. Generally, macromers have a molecular weight of about 500 Da or greater. In some modes of practice, macromers are used as a primary component in the hydrogel-forming composition and have a molecular weight in the range of about 1000 Da to about 2.times.10.sup.6 Da. Any type of macromer can be included in the hydrogel-forming composition of the invention. The macromer can be based on a synthetic or a natural polymer. Generally, the macromer or macromers used are substantially or entirely non-biodegradable.

Exemplary macromers that can be used include synthetic macromers based on the following polymers: poly(vinylpyrrolidone) (PVP), poly(ethylene oxide) (PEO), poly(ethyloxazoline), poly(propylene oxide) (PPO), poly(meth)acrylamide (PAA) and poly(meth)acylic acid, poly(ethylene glycol) (PEG) (see, for example, U.S. Pat. Nos. 5,410,016, 5,626,863, 5,252,714, 5,739,208 and 5,672,662) PEG-PPO (copolymers of polyethylene glycol and polypropylene oxide), hydrophilic segmented urethanes (see, for example, U.S. Pat. Nos. 5,100,992 and 6,784,273), and polyvinyl alcohol (see, for example, U.S. Pat. Nos. 6,676,971 and 6,710,126).

The hydrogel-forming composition can also include a macromer that is based on a natural polymer. Exemplary natural polymers include polysaccharides and polypeptides. Naturally occurring polysaccharides include polysaccharide and/or polysaccharide derivatives that are obtained from natural sources, including plants, animals, and microorganisms. The naturally occurring polysaccharide can be a homoglycan or a heteroglycan; exemplary heteroglycans include diheteroglycans and triheteroglycans. These naturally occurring polysaccharides can also be derivatized to provide pendent reactive groups that are members of a reactive pair, as described herein.

Desirably, the polysaccharide is highly hydrophilic and has the capacity of absorbing water when polymerized in the hydrogel in macromer form. Exemplary naturally occurring polysaccharides include dextran, hyaluronic acid, heparin, hydroxyalkyl cellulose, chondroitin sulfate, dermatan sulfate, heparan sulfate, keratan sulfate, dextran sulfate, pentosan polysulfate, chitosan, alginates, pectins, agars, glucomannans, and galactomannans.

In one aspect a hyaluronic acid (HA) macromer is included in the hydrogel-forming composition. Hyaluronic acid is a nonadhesive (to proteins), nonimmunogenic, and naturally derived linear polymer that includes alternating beta.1,4-glucuronic acid and beta.1,3-N-acetyl-D-glucosamine units. HA is the principal glycosaminoglycan in connective tissue fluids. Commercially available preparations of HA (such as HA Na.sup.+salt) can be used to prepare the macromer. Any sort of water-soluble HA polymer or water-soluble HA polymer derivative can be used as a macromer component in the present invention. Water-soluble esterified derivatives of HA, such as HAs having partial esterification, can be included in the matrix forming composition. For example, derivatives of HA such as benzyl esters of HA (Italiano, G. et al. (1997) Urol. Res., 25(2):137-42) can be used as macromers in the present hydrogel-forming compositions. In other aspects, low molecular weight fragments of HA (Chen and Abatangelo (1999) 30 Wound Repair Regen., 7:79-89) can be used as macromers in the present matrix-forming compositions.

Hyaluronic acid can be obtained from eukaryotic sources such as bovine vitreous humor, rooster combs, or umbilical cords, and also can be obtained from bacterial sources such as Streptococcus zooepidemicus. Depending on the desired use for a polymerizable composition that includes HA, one or more of these sources can be used for the preparation of the composition.

In many aspects, the hydrogel can be formed using polymers, such as macromers or polymers having pendent reactive pairs, at a total concentration of about 50% or greater, which allows for the formation of a hydrogel having at least moderately firm properties and suitable for use within the joint. More desirably the total concentration of macromer is 75% or greater, and concentrations of about 90% or greater have been found to produce a relatively firm gel.

However, in some aspects a lower concentration of polymer may be used. For example, if supporting tissue such as an annulus surrounds the pillow, the hydrogel may have a lower strength and modulus. In these aspects, for example, the total concentration of polymer can be less than 50%.

The hydrogel-forming composition can also include monomers that provide the formed hydrogel with hydrophobic segments. The hydrophobic segments can regulate the amount of water drawn into the hydrogel and provide strength to the hydrogel. U.S. Pub. No. 2006/0093648 describes hydrogels that are useful within joints. Hydrophilic macromers, such as PEG macromers, can be copolymerized with amphiphilic monomers such as diacetone acrylamide (DAA), vinyloxyethanol (VOE), 2-acrylamido-2-methylpropane (AMPS), and methyl acryloyl lactate (ALM) and its relatives.

In another aspect, the hydrogel-forming composition can also include a blend of two or more different macromers. In some aspects, the composition includes a first macromer that is highly hydrophilic and provides significant water retention properties to the hydrogel, and a second macromer that has a low molecular weight and provides the hydrogel with a relatively high modulus. In some aspects the first macromer comprises a plurality of pendent charged groups. For example, the charged groups can be selected from the group of carboxylate, amine, and sulfhydryl groups. The second macromer has a low molecular weight, desirably about 5000 Da or less, or about 2500 Da or less. One exemplary mixture includes a HA macromer and a PEG macromer.

In some aspects the hydrogel-forming composition includes the first (highly hydrophilic) macromer at a concentration of about 1% or greater and a second (low molecular weight) macromer at a concentration of about 40% or greater. An exemplary range for the first macromer is from about 1% to about 10%. An exemplary range for the second macromer is from about 40% to about 80%.

An exemplary composition includes a HA macromer at a concentration of about 10% and a PEG macromer at a concentration of about 50%.

The hydrogel-forming composition can also include one or more other ancillary reagent(s) that help promote formation of the hydrogel following delivery of the composition to the casing. These reagents can include polymerization co-initiators, reducing agents, and/or polymerization accelerants known in the art. These ancillary agents can be included in the composition at any useful concentration.

Exemplary co-initiators include organic peroxides, such as those that are derivatives of hydrogen peroxides in which one or both of the hydrogen atoms are replaced by an organic group. Organic peroxides contain the —O—O— bond within the molecular structure, and the chemical properties of the peroxides originate from this bond. In some aspects of the invention, the peroxide polymerization co-initiator is a stable organic peroxide, such as an alkyl hydroperoxide. Exemplary alkyl hydroperoxides include t-butyl hydroperoxide, p-diisopropylbenzene peroxide, cumene hydroperoxide, acetyl peroxide, t-amyl hydrogen peroxide, and cumyl hydrogen peroxide.

Other polymerization co-initiators include azo compounds such as 2-azobis(isobutyronitrile), ammonium persulfate, and potassium persulfate.

As discussed herein, the polymeric material may comprise a biodegradable polymer, a non-biodegradable polymer, or a combination thereof. Examples of polymers and macromolecule therapeutic agents include each and every one of the polymers and agents identified above.

The drug delivery systems of the present invention may be inserted into the eye, for example the vitreous chamber of the eye, by a variety of methods, including intravitreal injection, such as with pre-filled syringes in ready-to-inject form for use by medical personnel. The location of the system may influence the concentration gradients of therapeutic component or drug surrounding the element, and thus influence the release rates (e.g., an element placed closer to the edge of the vitreous may result in a slower release rate). The hydrogel suspensions can be administered via standard known needles, such as 27 g or 30 g needles, delivering up to about 1.5 mg siRNA per dose, depending upon the condition to be treated.

The present systems are configured to release an amount of the therapeutic agent effective to treat or reduce a symptom of an ocular condition, such as an ocular condition such as glaucoma or edema. More specifically, the systems may be used in a method to treat or reduce one or more symptoms of glaucoma or proliferative vitreoretinopathy.

In one embodiment, the nanoparticles of the invention are administered to a posterior segment of an eye of a human or animal patient, and preferably, a living human or animal. In at least one embodiment, such administration is performed without accessing the subretinal space of the eye. For example, a method of treating a patient may include placing the nanoparticles of the invention are directly into the posterior chamber of the eye. In other embodiments, a method of treating a patient may comprise administering the nanoparticles of the invention to the patient by at least one of intravitreal injection, subconjuctival injection, subtenon injections, retrobulbar injection, and suprachoroidal injection.

In at least one embodiment, a method of reducing neovascularization or angiogenesis in a patient comprises administering one or more nanoparticles of the invention containing one or more therapeutic agents, as disclosed herein to a patient by at least one of intravitreal injection, subconjuctival injection, sub-tenon injection, retrobulbar injection, and suprachoroidal injection.

In another aspect of the invention, kits for treating an ocular condition of the eye are provided, comprising: a) a container comprising an extended release implant comprising a therapeutic component including a therapeutic agent as herein described, and a drug release sustaining component; and b) instructions for use. Instructions may include steps of how to handle the drug delivery system of the invention, how to administer the drug delivery system of the invention into an ocular region, and what to expect from using the implants.

Use of nanoparticles in the invention avoids problems associated with sedimentation and, as compared to micorspheres or larger implants, provides a system for suitable therapeutic treatment, even at higher dosages, without any undesirable visual impairment. Administration of nanoparticles also permits multipoint administration of the encapsulated therapeutic agent into the ocular region, thereby avoiding dosage gradients that may be associated with larger implants.

### 3. EXAMPLES

The following non-limiting examples provide those of ordinary skill in the art with specific preferred drug delivery systems and methods for making such systems.

3.1. Manufacture and Testing of Nanoparticles Comprising a Therapeutic siRNA and a Biodegradable Polymer Matrix

In one embodiment of the delivery systems of the present invention, the nanoparticles are manufactured by a double emulsion process in which siRNA is dissolved in water and first emulsified in a chloroform solution comprising chloroform and matrix polymer to make a primary emulsion. The primary emulsion is added to PBB buffer comprising 2% polyvinyl alcohol (PVA) and homogenized at high speed. The chloroform is removed by evaporation in a vacuum, and the particles are washed by repeating one or more cycles of pelleting by ultracentrifugation and resuspending in PBS buffer. The so-created nanoparticles are then resuspended in solution.

The nanoparticles of the drug delivery systems of the instant invention may further comprise one or more pharmaceutically acceptable carriers, such as preservatives, salts, buffers, etc. When one or more pharmaceutically acceptable carrier is included in the nanoparticles of the present invention, the pharmaceutically acceptable carrier is added either before, during or after the above-described manufacturing process. For instance, a pharmaceutically acceptable carrier may be included in nanoparticles of the instant invention by dissolving, mixing, etc. into the siRNA—water solution, the chloroform solution, the primary emulsion and/or the PBB—PVA solution.

Alternatively, one or more pharmaceutically acceptable carriers can be introduced into formed nanoparticles by diffusion, electrophoresis and other techniques. A person of ordinary skill in the art will take into account the size and chemical properties of the pharmaceutically acceptable carrier to be included in nanoparticles of the instant invention as well as the size and chemical properties of the polymer matrix and siRNA of the nanoparticles in determining and appropriate time and to include any pharmaceutically acceptable carrier in nanoparticles of the instant invention.

The drug delivery systems can be sterilized by gamma irradiation. As an example, the nanoparticles can be sterilized by 2.5 to 4.0 mrad of gamma irradiation in their final primary packaging system, such as an administration device (e.g. a syringe applicator). Alternatively, the nanoparticles can be sterilized alone and then aseptically packaged into an sterile applicator system. In this case, the system can be sterilized by gamma irradiation at low temperatures to improve stability or blanketed with argon, nitrogen or other means to remove oxygen. Beta irradiation or e-beam irradiation or UV irradiation may be used. A dose of radiation from any source can be adjusted depending on the initial by a burden of the drug delivery systems.

Alternatively, the drug delivery systems may be manufactured under aseptic conditions from sterile starting components. The starting components may be sterilized by heat, irradiation (gamma, beta, UV, etc.), filtration, etc. The sterilized starting components are then used to aseptically produce the sterile drug delivery systems.

Nanoparticles manufactured according to this process can be analyzed for particle size and particle surface zeta potential by known methods. For instance, the nanoparticles can be dissolved in chloroform and extracted with an equal volume of phosphate buffered saline (PBS) buffer. The amount of polymer dissolved in chloroform can be measured by weighing after chloroform evaporation, and the amount of siRNA in the PBS solution can be measured by HPLC, photospectrometry, etc.

3.2 Nanoparticles Comprising siRNA027

Exemplary nanoparticles are manufactured according to the instant manufacturing method. In particular, siRNA027 was dissolved in water and emulsified in a chloroform solution comprising chloroform and matrix polymer PLGA to make the primary emulsion. The primary solution is then added to PBB comprising PVA, and homogenized in a homogenizer. The so-created nanoparticle solution is then pelleted in an ultracentrifuge, and resuspended in PBS. The resuspended nanoparticles are pelleted and resuspended. This manufacturing method provided nanoparticles of 350 nm, with a zeta potential of 1.3 mV and an siRNA drug content of 2.6% (w/w).

The present invention also encompasses the use of any and all possible combinations of the therapeutic agents disclosed herein in the manufacture of a medicament, such as a drug delivery system or composition comprising such a drug delivery system, to treat one or more ocular conditions, including those identified above.

All references, articles, publications and patents and patent applications cited herein are incorporated by reference in their entireties.

While this invention has been described with respect to various specific examples and embodiments, it is to be understood that the invention is not limited thereto and that it can be variously practiced within the scope of the following claims.

What is claimed is:

1. An intraocular drug delivery system comprising at least one short interfering ribonucleic acid (siRNA) molecule encapsulated in a nanoparticle comprised of a polymeric component comprised of at least one biodegradable polymer, a biodegradable copolymer or combinations thereof.

2. The system of claim 1, wherein the polymeric component comprises at least one member selected from the group consisting of a poly-lactic acid (PLA), poly-glycolic acid (PGA), poly-lactide-co-glycolide (PLGA), polyesters, poly (ortho ester), poly(phosphazine), poly (phosphate ester), polycaprolactones, gelatin, collagen, polyethyleneglycol (PEG), triblock copolymers, poly(D,L lysine) and derivatives thereof.

3. The system of claim 2, wherein the siRNA is encapsulated with the polymeric component in free form or in complex with a cationic polymer.

4. The system of claim 3, wherein the cationic polymer is a protamine.

5. The system of claim 1, wherein the siRNA inhibits cellular production of a urokinase, vascular endothelial growth factor, vascular endothelial growth factor 165 or vascular endothelial growth factor receptor.

6. The system of claim 1, wherein the siRNA has at least one property selected from the group consisting of an anti-bacterial agent, anti-angiogenic agent, anti-inflammatory agent, neuroprotectant agent, growth factor inhibitor agent, intraocular pressure reducing agent and ocular hemorrhage therapeutic agent.

7. The system of claim 1, wherein the size of said nanoparticles is between 100 nm and 200 nm, and wherein said nanoparticles have low polydispersity.

8. The system of claim 1, wherein the nanoparticles are comprised in an aqueous suspension.

9. The system of claim 8, wherein the aqueous suspension is contained in a viscoelastic hydrogel.

10. The system of claim 9, wherein the viscoelastic hydrogel comprises a high molecular weight hyaluronic acid.

11. The system of claim 10, wherein the nanoparticles comprise up to 30% w/w of the viscoelastic hydrogel, and wherein the hyaluronic acid comprises between 1% and 5% of the viscoelastic hydrogel.

12. A method of producing a sustained-release intraocular drug delivery system which comprises encapsulating siRNA molecules in nanoparticles comprised of at least one biodegradable polymer, a biodegradable copolymer or combinations thereof.

13. The method of claim 12, wherein the nanoparticles are between 100 and 200 nm.

14. The method of claim 13, wherein the formulation comprises an aqueous suspension of said nanoparticles.

15. The method of claim 14, wherein the aqueous suspension comprises a sterile viscoelastic hydrogel.

16. The method of claim 15, wherein the viscoelastic hydrogel comprises high molecular weight hyaluronic acid.

17. The method of claim 16, wherein the nanoparticles comprise up to 30% w/w of the viscoelastic hydrogel, and wherein the hyaluronic acid comprises between 1% and 5% of the viscoelastic hydrogel.

18. A method for treating an ocular condition which comprises administering to the ocular area of a patient, and effective amount of a drug delivery system according to claim 1.

19. The method of claim 18, wherein the ocular condition includes retinal damage.

20. The method of claim 18, wherein the ocular condition is glaucoma, proliferative vitreoretinopathy, or age related macular degeneration.

21. The method of any one of claims 18-20, wherein the system is administered via injection into the eye.
